(19) United States
(12) Patent Application Publication
(10) Pub. No.: US 2004/0087655 A1
(43) Pub. Date: May 6, 2004

(54) 6-Substituted indanoyl amino acid conjugates as mimics to the biological activity of coronatine

(76) Inventors: Gode Schuler, Dusseldorf (DE); Wilheim Boland, Durmerstein (DE); Ryan Lauchu, Irvine, CA (US)

Correspondence Address: FOLEY AND LARDNER, SUITE 500, 3000 K STREET NW, WASHINGTON, DC, 20007, US

(21) Appl. No.: 10/466,361

(22) PCT Filed: Jan. 16, 2002
(86) PCT No.: PCT/EP02/00405

(30) Foreign Application Priority Data
Jan. 16, 2001 (EP) 01100171.6

(51) Int. Cl. A61K 31/198; C07C 235/32; C07C 251/32

(52) U.S. Cl. 514/563; 560/41; 562/450

(57) ABSTRACT

Described are 6-substituted indanoyl amino acid conjugates as defined in claim 1 of the present invention as potent plant elicitors and efficient mimics of the phytotoxin coronatine. Also, processes for producing the improved elicitors are provided, which allow a rapid and convenient access to large quantifies of the highly active compounds. Furthermore, there are compositions and plant protecting agents described, comprising as active ingredient a compound of the present invention. The plant protecting agents are useful for inducing resistance to pathogens in plants. The compositions may also be used to selectively induce senescence in fruit of plants.

The invention relates to 6-substituted indanoyl amino acid conjugates which act as mimics of the naturally occurring compound coronatine. The compounds show superior properties as plant elicitors as compared to compounds known from the prior art. The invention also relates to a process for producing the compounds of the present invention with a high efficacy and yield. The invention furthermore deals with the use of the compounds for inducing resistance in plants against pathogens and to plant protecting agents comprising the 6-substituted indanoyl amino acid conjugates.

In recent years the phytotoxin coronatine with the structural formula A (below) attracted considerable interest, since coronatine mimics many of the biological activities generally associated with jasmonic acid (the structural formula of which is shown as formula B below), a powerful low molecular weight signalling molecule involved in plant stress responses.

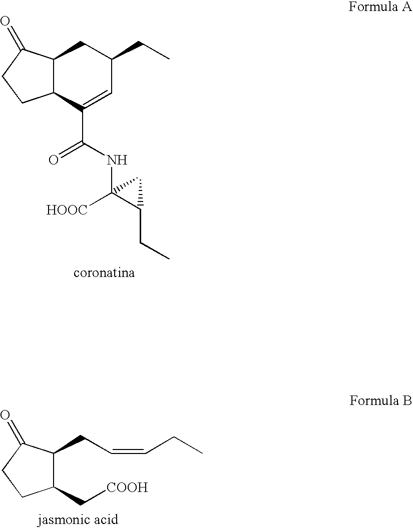

Coronatine Is a conjugate of the non-aromatic polyketide coronafacic acid with the rare cyclopropyl amino acid coronamic acid. It is a well investigated plant elicitor. The phytotoxin is produced by several pathovars of Pseudomonas syringae (e.g. tomato, glycinea, atropurpurea) and was first isolated by Ichihara et al. in 1978 (J. Am. Chem. Soc. 1977, 99, 636-637) from a fermentation broth of P. syringae var. atropurpurea.

Application of coronatine to higher plants elicits a spectrum of responses, especially diffuse chlorosis, tendril coiling in Bryonia diocia, emission of ethylene, as well as the biosynthesis of terpenoids and other volatiles that are often involved in the complex network of signalling among plants, herbivores, and their parasites. The compound apparently bypasses the activation of the lipid-based signalling pathway by interacting directly with the receptors or binding proteins of the genuine signals such as 12-oxo-phytodienoic acid and/or jasmonic acid.

Interestingly, and important for practical applications, in most assays coronatine proved to be much more active than did 12-oxo-phytodienoic acid and/or jasmonic acid.

For example, coronatine stimulates the production of the antitumor active paclitaxel (also known under the trademark Taxol®) in cell cultures of Taxus media more efficiently than jasmonic acid or methyl jasmonate (JAMe). Other examples for inducible products are phytoalexins from rice.

In addition to coronatine, several other conjugates of coronafacic acid with, for example, norcoronamic acid, L-isoleucine, and L-valine have been isolated and were found to be biologically active. Also, several microbial- or insect derived high- and/or low-molecular-weight metabolites been investigated as their ability to induce the biosynthesis of volatiles in plants. It is, however, often difficult to produce such compounds in larger scales.

Due to this scientific and economically interesting profile of the biological activities of coronatine, there is a need to design compounds which act as mimics of coronatine. Also, such compounds should be easily available by an efficient and high-yielding chemical synthesis.

In the prior art, attempts have already been made to find analogues to coronatine. As one functional analogue of coronatine, the 1-oxo-indanoyl-isoleucine methyl ester has been investigated. That compound is not substituted in the 6-position of the indanoyl moiety. Like coronatine, the aromatic analogue is an elicitor of plant secondary metabolism. The spectrum of the biological effects of 1-oxo-indanoyl-isoleucine methyl ester studied so far, however, was clearly more jasmonate-related than that of coronatine. The latter stimulates a broader range of biological responses.

Starting from 1-oxo-indanoyl-isoleucine methyl ester, a photoreactive conjugate for tagging binding proteins and/or receptors was developed. The introduction of the photolabile azido group at C-6 of the indanoyl moiety modified the biological activity of the resulting compound (volatile induction) more in the direction of coronatine. However, the 6-azido-1-oxo-indanoyl isoleucine derivative is unstable, in particular in solution. It rapidly degrades upon contact with light and is therefore is only useful as a plant elicitor in artificial systems, i.e. under strict dark conditions.

Therefore, the technical problem underlying the present invention to is overcome the drawbacks of the compounds used according to the prior art as plant elicitors and coronatine mimics and to provide new and advantageous compounds being mimics of coronatine with improved biological activity. It is a further object to provide an easy and economic process leading to high yields for producing such compounds.

This problem is solved by the provision of the embodiments as characterised in the claims.

Accordingly, the present invention relates to compounds of the following chemical formula I:

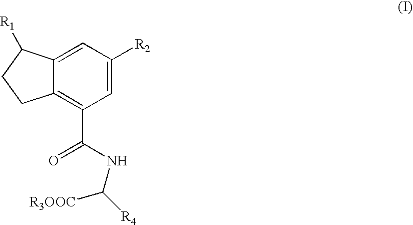

wherein

R₁=

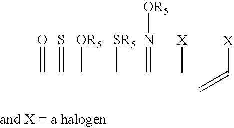

atom;

R₂=linear or branched C₁-C₈-alkyl, -alkenyl or -alkinyl; saturated or unsaturated, linear or branched C₁-C₈-ether or -polyether, or

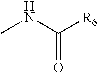

R₃=H or a residue that forms an ester which can be easily saponified by the plant;

R₄=a side chain of an L-amino acid;

R₅=H, acyl, or a linear or branched C₁-C₈-alkyl, -alkenyl, or -alkinyl; and

R₆=H or a linear or branched C₁-C₈-alkyl, -alkenyl, or -alkinyl; or a saturated or unsaturated, linear or branched C₁-C₈-ether or -polyether.

In accordance with the invention, it has surprisingly been found that the substituent characterised as R₂ in the 6-position of the indanoyl moiety leads to a much better activity profile of the resulting plant elicitor as compared to the respective prior art compound being not substituted in that position. Furthermore, the described compounds have the advantage that they can be produced in an easy manner and are therefore suited for production in an industrial scale.

Preferred as R₂ In the above formula are linear or branched C1-C8 alkyl residues, such as methyl, ethyl, n- or i-propyl, butyl, linear or branched C2-C8 alkenyl residues such as allyl, propenyl, butenyl, linear or branched C1-C8 alkoxy residues, such as methoxy, ethoxy, propoxy, butoxy, pentoxy or unsaturated C₂-C₈ alkenoxy residues such as allyloxy, propenoxy, etc. or cyclic or heterocyclic structures such as furanyloxy.

As to the residue R₃, the biological active elicitor is the free acid, i.e. a compound wherein R₃ is H. Useful as R₃ is therefore any residue which forms an ester that can be saponified by the plant, i.e. the respective ester can be easily converted into the free acid by the plant Non-limiting examples of such residues are linear or branched C₁-C₄-alkyl, -alkenyl, or -alkinyl residues, benzyl, phenyl or allyl.

As to the residue R₄, said residue is defined to be the side chain of an L-amino acid, i.e. the residue attached to the central carbon atom of an amino acid. In other words, the carbon atom to which R₄ is attached may be considered as the α-C atom of an amino acid or amino acid ester residue formed by this C-atom together with R₄, the carboxyl residue including R₃ and the amino group forming the amide bond with the substituted indanoyl moiety of formula (I). In the sense of the invention, the term amino acid encompasses all naturally occurring amino acids including the rare amino acids. In particular suited are aliphatic or allo-isoleucin, norvalin, norleucin or coronamic acid and its biochemical precursors isocydic amino acids.

In a preferred embodiment of the invention, R₄ is the side chain of isoleucin, leucin,

A particular preferred plant elicitor according to the present invention is 6-ethyl-1-oxo-indanoyl isoleucine methyl ester (according to the IUPAC nomenclature: 2-[(6-Ethyl-1-oxo-indane-4-carbonyl)-amino]-3-methyl-pentanoic acid methyl ester).

In the following, the improved properties of the compounds of the present invention, in particular with respect to the prior art compound 1-oxo-indanoyl-isoleucine methyl ester, will be described in more detail:

To assess the profile of activities of a plant elicitor, the analysis of a blend of induced volatiles is of particular value since the spectrum of emitted compounds comprises many metabolites from very different pathways. Since a complex network of signals individually regulates the different pathways, differences in the elicitor-activity of test compounds may be reflected in qualitative and/or quantitative composition of the volatile blends.

Upon investigation of the compounds of the present invention as compared to the prior art compound 1-oxo-indanoyl-isoleucine methyl ester (which is not substituted in the 6-position of the indanoyl moiety), the analysis of the volatile blend shows the superior properties of, for example, the novel compound 6-ethyl-1-oxo-indanoyl isoleucine methyl ester.

In contrast to prior art compound being unsubstituted at the 6-position of the indanoyl moiety, the novel elicitor proved to be ca. 30-50 fold more active, with a threshold concentration of less than 10 μM. Significant qualitative and quantitative differences of volatiles elicited by the two compounds become apparent from FIG. 2.

For example, methyl salicylate and the C₁₆ terpenoid TMTT (see FIG. 2) were induced only by treatment with 6-ethyl-1-oxo-indanoyl isoleucine methyl ester and coronatine. The C₁₁ terpenoid hydrocarbon 4,8-dimethyl-nona-1,3,7-triene (DMNT) is present in both volatile blends, but the up-regulation of its biosynthesis is much more pronounced after treatment with the 6-substituted compound according to the invention.

In summary, the volatile blend resulting from elicitation with 6-ethyl-1-oxo-indanoyl isoleucine methyl ester is closer related to the effect of coronatine than to that of the unsubstituted prior art compound or jasmonic acid.

Unlike coronatine, the 6-substituted amino acid conjugates according to the invention can be applied as esters (reactive methyl- and allylesters are preferred) since phytogenic esterases are apparently able to generate the free acids required for elicitation.

The emission of significant amounts of methyl salicylate after stimulation with 6-ethyl-1-oxo-indanoyl isoleucine methyl ester suggests that the novel elicitor may also be able to enhance the plant's resistance, which is generally mediated by salicylate.

Indeed, the analysis of the internal level of salicylic acid in plants pre-treated with 6-ethyl-1-oxo-indanoyl isoleucine methyl ester confirmed a strongly enhanced level of salicylic acid (8 fold increase of salicylate).

Like coronatine, 6-ethyl-1-oxo-indanoyl isoleucine methyl ester is also a potent elicitor of the coiling reaction of touch-sensitive tendrils of Bryonia dioica. The coiling reaction is induced by coronatine with a threshold concentration of about 2 μM. However, to induce a response with the prior art compound being unsubstituted in the 6-position, rather high concentrations in the range of about 1 mM were necessary. In contrast to prior art compound, the 6-ethyl-1-oxo-indanoyl isoleucine methyl ester according to the invention induced a coiling response already at a concentration of 20 μM.

In addition to the induction of volatile biosynthesis in the lima bean and tendril coiling in Bryonia dioica, the compounds of the present invention trigger a number of additional responses in other plants. They are efficient mimics of the phytotoxin coronatine. The introduction of a substituent at C-6 of the indanoyl moiety strongly enhances the activity of the compounds (ca. 30-50 fold with respect to the unsubstituted prior art compound) and at the same time tunes the activity profile of the compounds more to that of coronatine.

The present invention is also related to a process for producing the 6-indanoyl substituted compounds as described above, according to which a 6-substituted 1-oxo-indan-4-carboxylic acid with the following structural formula (II)

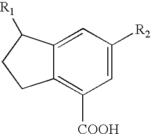

wherein

R₁=

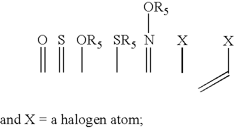

R₂=linear or branched C₁-C₈-alkyl, -alkenyl, or -alkinyl; saturated or unsaturated, linear or branched C₁-C₈-ether or -polyether or

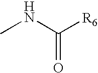

R₅=H, acyl, or linear or branched C₁-C₈-alkyl, -alkenyl, or -alkinyl;

R₆=H or linear or branched C₁-C₈-alkyl, -alkenyl, or -alkinyl; or a saturated or unsaturated, linear or branched C₁-C₈-ether or -polyether

is reacted with an L-amino acid or amino acid ester.

Preferred embodiments of R₁, R₂, R₄, R₅ and R₆ in formula (II) are as defined for the corresponding residues of formula (I) above.

The process leads to a reaction between the amino group of the amino acid or the respective ester with the —COOH group of the carboxylic acid. The reaction conditions for carrying out such a process are known to the person skilled in the art and are for example described in Speicher et al., J. Prakt. Chem., 1998, 340, 581-583.

The process enables a rapid and convenient access to large quantities of the above described compounds:

Preferably, the reaction is carried out in a mixture of DMF and collidine in the presence of (O-(7-Aza-1-benzotriazolyl)-N,N,N′,N′-tetramethyluroniumhexafluoro-phosphate (HATU).

As explained before, the amino acid or its ester may be any L-amino acid, in particular any naturally occurring amino acid. In particular suited are aliphatic or isocyclic amino acids. Preferred are isoleucin, leucin, allo-isoleucin, norvalin, norleucin or coronamic acid and its biochemical precursors.

In a first preferred embodiment of the process according to the invention, the compound of formula (II) to be reacted with the amino acid is synthesised according to a reaction scheme comprising the following steps:

a) reacting tetrahydronaphthalin in the presence of AlCl₃ and an acyl halide R′-C(O)X to form a diketone (2)

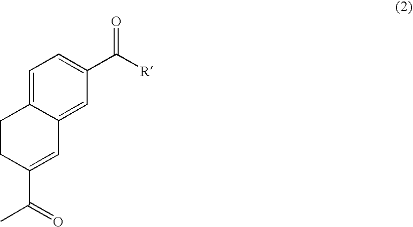

b) oxidative cleavage of (2) at the non-aromatic double bond to yield the triketone intermediate (2′)

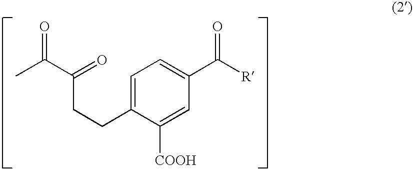

which is rapidly further oxidised to give the dicarboxylic acid (3)

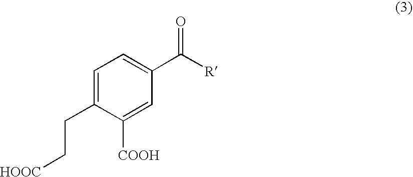

c) reduction of (3) to yield the aromatic dicarboxylic acid (4)

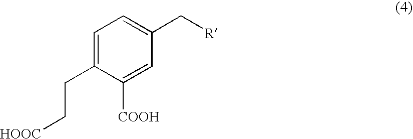

d) and effecting an intramolecular Friedel-Crafts acylation on (4) to to yield a compound of formula (II), wherein R₁ is ═O and R₂ is a linear or branched C2-C8-alkyl, alkenyl, or alkinyl residue.

The acyl halides R′-C(O)X used for this purpose are preferably chosen so as to provide, after reduction in step c), the residue R₂ in the target molecule of formula (I) or (II) as a C1-C8-alkyl, alkenyl, or alkinyl group. Accordingly, R′ in the above formula is hydrogen or a linear or branched C1-C7-alkyl, alkenyl, or alkinyl residue, such as ethyl, propyl or butyl, and X is a halide, preferably chloride or bromide.

According to usual practice know from the art, 1-Oxo-indan-4-carboxylic acid (the original mimic of coronafacic acid) is available by intramolecular Friedel-Crafts acylation of 2-(2-carboxy-ethyl)-benzoic acid. Starting from 1-Oxo-indan-4-carboxylic acid, the prior art compound 1-oxo-indanoyl-isoleucine methyl ester can be obtained. However, attempts to functionalise the aromatic nucleus of 1-oxo-indanoyl-isoleucine at C-6 via bromination, nitration, acylation etc. failed owing to the strong deactivation by the two adjacent carbonyl groups.

The preferred process according to the invention is an efficient alternative approach, providing directly the required substitution pattern of the aromatic nucleus by the conversion of tetrahydronaphthalin. This synthetic approach is flexible and advantageous as compared to prior art processes. It allows the introduction of different substituents at C-6 of the indanoyl moiety, leading to elicitors with different activity profiles. Furthermore it allows the use of low-cost educts, thereby providing an economic means for producing the described compounds.

In particular, the aromatic analogue of coronafacic acid (5)

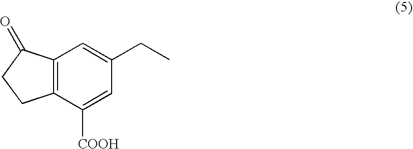

is available in only four simple operations and has a good overall yield. All transformations can be easily performed on a large scale (ca. 10 g) and do not require dedicated reaction conditions or reagents. The keto-functional group at the indanon part of (5) can be easily substituted by any desired residue within the definition of R₁ prior to the final coupling reaction with an amino acid.

Conjugation of the indanoyl carboxylic acid (5) with the hydrochloride of an desired amino acid or its ester, depending on which residues are desired for R₃ and R₄ in the final product, is best achieved in DMF using HATU (O-(7-Aza-1-benzotrlazolyl)-N,N,N′,N′tetramethyluroniumhexafluoro-phosphate) for activation of the amino acid and the indanoyl moiety.

Particularly preferred is a process following Scheme 1 below

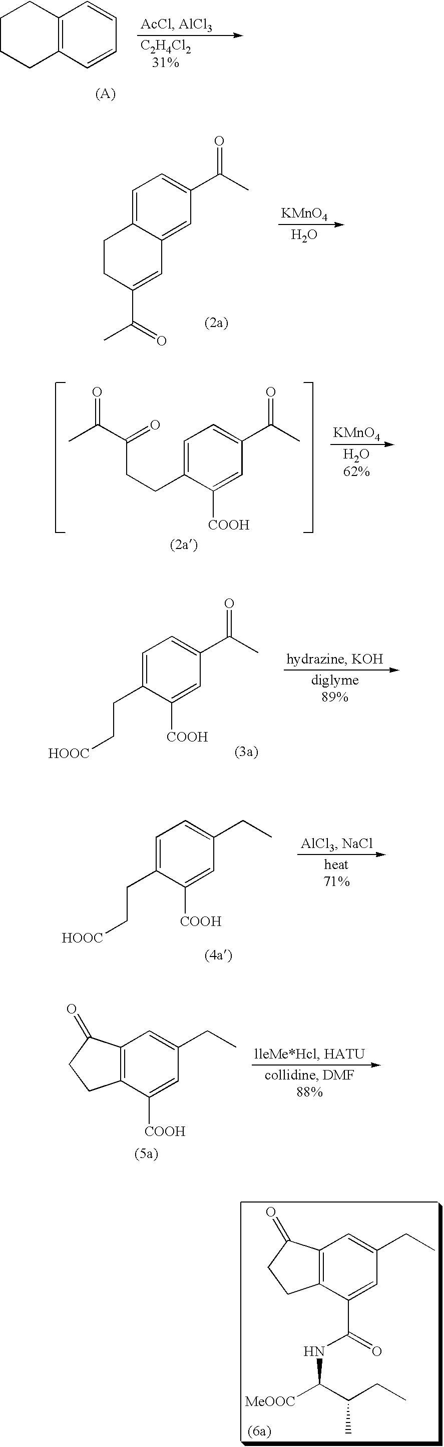

In Scheme (1), tetrahydronaphthalin (tetralin (1)) is reacted in the presence of AlCI₃ and acetyl chloride to the diketone (2 a).

Subsequent oxidative cleavage of (2a) with aq. KMnO₄ proceeds smoothly with exclusive cleavage of the non-aromatic double bond to yield the triketone intermediate (2′a), which is rapidly further oxidised to give the dicarboxylic acid (3a).

The second, rapid cleavage reaction of the vicinal diketone (2′a) is of high synthetic value since it removes the complete carbon skeleton of the second acylation step at the saturated ring moiety, resulting only in the C-5 substituted aromatic nucleus with the correct substitution pattern required for the envisaged intramolecular Friedel-Crafts cyclisation.

The sequence (1)→(3a) is generally applicable, since substitution of (1) proceeds with many desired acyl halides by analogy. For example, acylation of (1) with butanoyl chloride and subsequent oxidative cleavage with KMnO₄ provides a dicarboxylic acid of type (3) in high yield bearing an 1-oxobutyl group at C-5 (which finally leads to an end product wherein R₂ is defined as propyl).

Attempts to achieve an intramolecular Friedel-Crafts acylation with (3a) failed owing to the deactivating effect of the keto group. However, after reduction of the carbonyl group with hydrazine in boiling triglyme, the resulting 5-substituted dicarboxylic acid (4a) could be smoothly cyclized to give the indanoyl carboxylic acid (5a). Heating with AlCl₃/NaCl furnished the 6-substituted 1-oxo-indan-4-carboxylic acid (5a) in 71% yield.

Conjugation of the indanoyl carboxylic acid (5a) with the hydrochloride of an desired amino acid or its ester, depending on which residues are desired for R₃ and R₄ in the final product, was achieved in DMF using HATU

Using the methyl ester of isoleucine leads to the final product (6a) in a 89% yield. Work-up, and purification of the product were superior to a previous protocol. The final 6-ethyl-1-oxo-indanoyl isoleucine methyl ester (6a) crystallised as monoclinic colourless prisms.

An alternative, preferred embodiment of the process according to the invention, leading to compounds of formula (I) carrying an ether bond at their 6-position is explained in the following. In this approach, oxidative cleavage of the non-aromatic double bond of 6-methoxy-1,2-dihydronaphtalene (7)

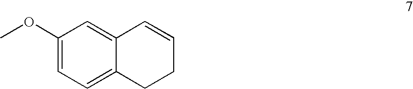

yields a dicarboxylic acid (8)

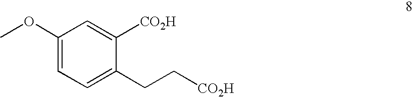

that is converted via intramolecular Friedel-Crafts acylation to give a 6-substiuted indanone derivative (9) as an intermediate, which can be used in further steps without being isolated.

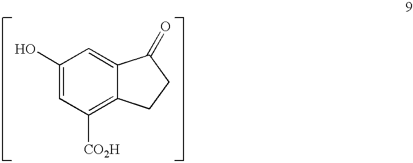

Formation of an ether of the 6-hydroxy group of the substituted indanone with RX, wherein X is a suitable leaving group such as a halide or a sulfonate residue and R is a linear or branched, saturated or unsaturated C1-C8 residue, optionally interrupted by further O atoms, yields a compound of formula (II), wherein R₁ is =0 and R₂ is a saturated or unsaturated, linear or branched C₁-C₈-ether or -polyether.

In a preferred, convenient embodiment of the second process, ether formation of the phenolic OH group at the 6 position of the indanone (9) is carried out simultaneously with esterificarion of the carboxyl group at its 4-position with RX, yielding compound (10)

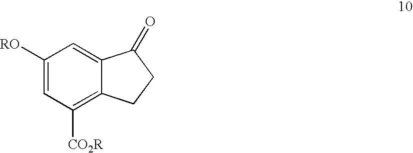

which is then subjected to saponification of the ester to arrive at the compound of formula (II).

The compound of formula (II) is subsequently reacted with an amino acid as generally described above, to yield a 6-substituted indanoyl amino acid conjugate of the present invention of formula (I).

Prior to the reaction with the amino acid, the keto-functional at the indanone part of compound (II) as obtained in this reaction sequence may be substituted by any desired residue further within the definition of R₁ in formulae (I) and (II).

Preferred embodiments of R in this second process are C1-C8 alkyl residues, such as methyl, ethyl, propyl, butyl or pentyl or C2-C8 alkenyl residues, such as allyl, propenyl, butenyl or pentenyl. Preferred leaving groups X, among those known in the art, are halides such as Br or I or sulfonate residues.

A particularly preferred embodiment of the process starting from 6-methoxy-1,2-dihydronaphtalene (7) to yield a compound of formula (I) is depicted in the following Scheme 2:

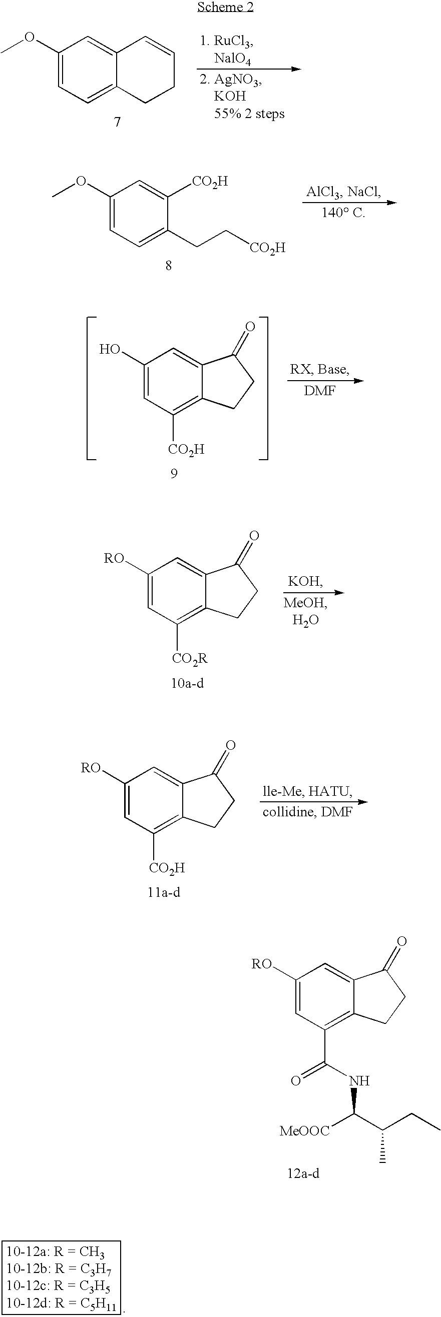

In the reaction sequence following Scheme 2,6-methoxy-1,2-dihydro-naphthalene (7) is used as the starting material. The double bond of 7 is readily cleaved with catalytic RuCl₃ and sodium periodate as the stoichiometric oxidant, leading to a mixture of aldehydes and carboxylic acids that is subjected to further oxidation with AgNO₃ and KOH. The desired dicarboxylic acid 8 can thus be obtained. Intramolecular Friedel-Crafts acylation can be achieved with simultaneous ether cleavage using a salt melt of AlCl₃ and NaCl at 140° C. The resulting crude product can be used immediately without isolation.

For, e.g., methoxy, propoxy, allyloxy, and pentoxy substituted indanoyl isoleucine conjugates, the strategy outlined in Scheme 2 can proceed as follows: the crude phenol 9 is sonicated in DMF with K₂CO₃ or Cs₂CO₃ along with the appropriate alkyl bromide and Kl or alkyl sulfonate, causing substitution by both the phenol and carboxylic acid groups.

Among others, propyl, allyl, and pentyl bromides can be used with success (10b-d), and dimethyl sulfate can be used for the synthesis of the 6-methoxy compound 10a since methyl iodide may lead to unwanted substitutions next to the carbonyl group of the indanone. The products created in this step are easily purified by chromatography. Hydrolysis with KOH yields the free carboxylic acids 11a-d. Conversion into the isoleucine methyl esters 12a-d can be achieved as described above for other compounds of formula (II).

The present invention also relates to a composition comprising a compound according to the invention. Such a composition is, for example, useful as a plant protection agent for treating plants to induce a resistance against pathogens. These pathogens are, for example but not limited to, harmful bacteria, fungi, viruses, insects and nematodes.

For inducing plants' resistance to pathogens, the composition or plant protecting agent are applied to the plant in a manner known to a person skilled in the art.

The compositions or plant protecting agents are usually in the form of a powder, a suspension, dispersion, emulsion, paste or granulate and may comprise furthermore an insecticide, a growth regulation agent, a herbicide, a fungicide and/or a fertiliser.

The present invention also relates to the use of a compound or a composition according to the invention for treating plants to induce a resistance against pathogens as well as to a method for treating a plant to induce a resistance against pathogens comprising the step of applying a compound, a composition or a plant protection agent according to the invention to the plant;

For treating the plants, the plant or crop protecting agents are preferably applied to the plant in such a way that the compounds of the invention are continuously distributed or dispersed onto the plant or its root.

In principle all types of plants can be treated according to the above described use or method according to the invention. Preferably, the plant is a useful plant, i.e. a plant which is cultured or used for nutritional or Industrial purposes. Most preferably the plant is a crop plant, such as e.g. cereals, vegetables etc.

The present invention also relates to the use of a compound or a composition according to the invention for inducing in plants senescence selectively in fruits but not In leaves. This can greatly facilitate harvest, e.g., in the case of plants producing citrus fruit, e.g. lemons, oranges or grapefruit.

Moreover, the present invention also relates to a method for selectively inducing senescence in fruits but not in leaves of plants comprising the step of applying a compound and/or composition according to the Invention to a plant. For the formulation and possibilities of applying the compound or composition the same holds true which was already described above in connection with the induction of resistance to pathogens. Plants in which senescence in fruits can be specifically induced can in principle be any fruit bearing plants, preferably plants producing citrus fruit, e.g. lemons, oranges or grapefruits. Furthermore, all embodiments characterised in the claims are herewith Incorporated.

### LEGENDS TO THE FIGURES

FIG. 1: Two pairs of symmetry independent molecules of the 6-ethyl-1-oxo-indanoyl isoleucine methyl ester are jointly packed into a single unit cell. The two aromatic systems are fixed In a sandwich-like fashion and kept in anti-orientatlon by two hydrogen bridges between the weakly acidic amide —N—H and the lone pairs of the oxygen atoms of the keto groups.

FIG. 2: Volatile blends emitted from leaves of the Lima bean P. lunatus after treatment with the 6-ethyl conjugate 6-ethyl-1-oxo-indanoyl isoleucine methyl ester (FIG. 2A) and the unsubstituted prior art compound (FIG. 2B). Identification of compounds: (a) β-ocimene, (b) linalool, (c) 4,8-dimetyl-nona-1,3,7-triene (DMNT), (d) C₁₀H₁₄, (e) methyl salicylate,

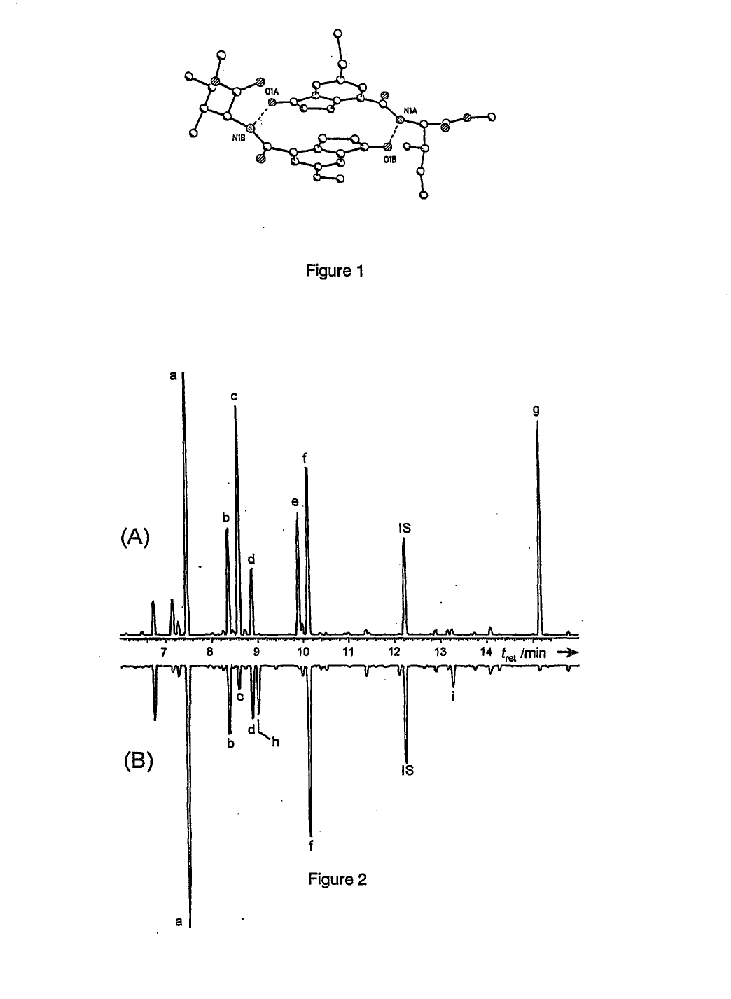

(f) C₁₀H₁₆O, IS: internal standard (1-bromodecane), (g) 4,8,12-trimethyltrideca-1, 3, 7, 11-tetraene (TMTT), (h) phenyl acetonitrile, (i) caryophyllene. Volatiles were collected by absorption onto carbon traps. Separation and identification of the compounds were achieved by GLC-MS.

The Invention will now be described by reference to the following examples which are merely illustrative and are not to be construed as a limitation of the scope of the present invention.

### EXAMPLES

General: Reactions were performed under protective gas (argon). Solvents were dried according to standard methods. Melting points were determined with Büchi B-540. IR: Bruker Equinox 55 FTIR Spectrophotometer. ¹H- and ¹³C NMR: Avance DRX 500 spectrometer; CDCl₃ or [D₆] DMSO as solvent. Chemical shifts of ¹H and ¹³C NMR are given in ppm (δ) downfield relative to TMS as internal standard. GC-MS (70 eV): Finnigan GCQ, equipped with a fused silica capillary, coated with DB5 (30 m×0.25 mm); helium served as carrier gas. HR-MS: Micromass MasSpec 2. Silica gel: Si 60 (0.200-0.063 mm, E. Merck, Darmstadt, Germany) was used for chromatography. Thin layer chromatography was performed with silica gel plates from Merck (60 F₂₅₆).

The numbering of the compounds occurring in brackets is Identical with the numbering used in the above reaction scheme.

### Example 1

### Production of 2-[(6-Ethyl-1-oxo-indane4-carbonyl)-amino]-3-methyl-pentanoic Acid Methyl Ester (6)

Step 1:1-(7-Acetyl-5,6-dihydro-naphthalen-2-yl)-ethanone (2): Acetyl chloride (19.0 g, 240 mmol) was added slowly at room temperature to a well-stirred solution of AlCl₃ (43.0 g, 320 mmol) in ethylene dichloride (20.0 ml) followed by slow addition of a solution of tetralin (tetrahydronaphthaline, (1), 10.0 g, 75.6 mmol) in C₂H₄Cl₂ (8.0 ml). When the vigorous reaction had ceased, the solvent was removed under reduced pressure, and the residue was heated to 100 C. for 2 h. Pure product was obtained by distillation at reduced pressure. B.p.: 190-200 C./8*10⁻3 bar. Yield: 5.1 g, 23.8 mmol (31%). The highly viscous oil solidified while standing to give a colourless crystalline solid. M.p.: 76° C.

¹H NMR (CDCl₃, 500 MHz): δ=2.36 (s, 3H), 2.50-2.55 (m, 5H), 2.80 (t, 2H), 7.20 (d, 1 H), 7.35 (s, 1H), 7.74 (s, 1H), 7.76 (d, 1H).-¹³C NMR (CDCl₃, 125 MHz): δ=20.5 (CH₃), 25.2 (CH₃), 26.4 (CH₂), 27.5 (CH₂), 127.9 (CHₐᵣₒₘ), 128.1 (CHₐᵣₒₘ), 129.6 (CHₐᵣₒₘ), 132.8 (C═C), 135.9 (Cₐᵣₒₘ), 136.0 (Cₐᵣₒₘ), 138.8 (C═C), 142.8 (Cₐᵣₒₘ), 197.1 (C═O), 198.1 (C═O).-IR (KBr):

v=3055, 3004, 2954, 2894, 2839, 1682, 1657, 1622, 1592, 1411, 1386, 1350, 1285, 1205, 833 xμ⁻¹.-MS (70 eV); m/z (%): 214 (100) [M⁺], 199 (80), 171 (82), 156(11), 141 (7), 128 (50).-HR MS [M⁺]: calcd. 214.0994; found 214.0990

Step 2: 5-Acetyl-2-(2-carboxy-ethyl)-benzoic acid (3): The diketone (2) (4.5 g, 21 mmol) was dissolved in the minimum amount of 1,2-dichloroethane (ca. 5 ml) and slowly added while stirring to a chilled aqueous solution of KMnO₄ (9.0 g, 57 mmol in 225 ml water). Stirring was continued at 0° C. for 3 h. Then NaOH (4.0 g, 100 mmol) was added and the solid MnO₂ was filtered off. The aq. solution was acidified with 12N HCl until pH 2 to precipitate the product (3). The dicarboxylic acid was filtered off and washed with a small amount of ice-cold ethanol (2 ml). Crystallisation from water afforded the pure dicarboxylic acid (3) as a colourless solid. Yield: 3.1 g, (62%). M.p.: 76° C.

¹H NMR ([D₆] DMSO, 500 MHz): δ=2.55 (t, J=7.7 Hz, 2H), 2.59 (s, 3H), 3.21 (t, J=7.7 Hz, 2H), 7.50 (d, J=8.2 Hz, 1H), 8.03 (d, J=8.2 Hz, 1H), 8.33 (s, 1H), 12.66 (s).-¹³C NMR ([D₆] DMSO, 125 MHz): β=26.7 (CH₂), 29.1 (CH₂), 34.9 (CH₃), 129.9 (CH_{arom ),} 130.9 (Cₐᵣₒₘ—COOH), 131.2 (CHₐᵣₒₘ), 131.3 (CHₐᵣₒₘ), 135.0 (CHₐᵣₒₘ—COCH₃), 147.1 (Cₐᵣₒₘ), 168.0 (COOH), 173.5 (COOH), 197.0 (C═O).-IR (KBr): v=3079, 3000, 2931, 1717, 1695, 1656, 1603, 1429, 1359; 1289, 1193, 1071, 835, 660 cm⁻1.-MS (70 eV); m/z (%): 236 (11) [M⁺], 221 (56), 218 (82), 203 (21), 190 (97), 175 (100), 147 (21), 91 (34), 77 (33), 65 (17).-HR MS [M⁺]: calcd 236.0685; found 236.0688.

Step 3: 2-(2-Carboxy-ethyl)-5-ethyl-benzoic acid (4): A suspension of the dicarboxylic acid (3) (3.0 g, 12.7 mmol), hydrazine hydrate (98%, 2 ml, 42 mmol)., finely powdered KOH (3.0 g, 53 mmol)) in triethylene glycol (75.0 ml) was refluxed for 2 h. Hydrazine and water were continuously removed by distillation until the temperature in the reaction flask remained constant at 195° C. After 4 h the suspension was allowed to cool to room temperature and the same volume of water was added. Acidification with conc. HCl precipitated the dicarboxylic acid (4). The product was extracted with ether (3×5 ml). Recrystallisation from water afforded the dicarboxylic acid (4) as a white solid. Yield: 2.5 g, 11.3 mmol (89%). M.p.: 174° C.

¹H NMR ([D₆] DSMO, 500 MHz): δ=1.17 (t, J=7.6 Hz, 3H), 2.50 (t, J=7.8 Hz, 2 H), 2.60 (q, J=7.6 Hz, 2H), 3.11 (t, J=7.8 Hz, 2H), 7.25 (d, J=7.8 Hz, 1H), 7.31 (d, J=7.8 Hz, 1H), 7.65 (s, 1H), 12.45 (s).-¹³C NMR ([D₆] DMSO, 125 MHz): δ=15.4 (CH₃), 27.5 (CH₂), 28.8 (CH₂), 35.4 (CH₂—COOH), 129.5 (CHₐᵣₒₘ), 130.3 (Cₐᵣₒₘ—COOH), 130.8 (CHₐᵣₒₘ), 131.2 (CHₐᵣₒₘ), 139.1 (CHₐᵣₒₘ—C2H5), 141.7 (Cₐᵣₒₘ), 168.8 (COOH), 173.8 (COOH).-IR (KBr): v=3031, 2967, 2933, 2638, 1688, 1403, 1305, 1275, 1211, 907, 828 cm⁻1.-MS (70 eV); m/z (%): 222 (3) [M⁺]; 204 (29), 176 (100), 159 (16), 148 (9).-HR MS [M⁺]: calcd 222.0892; found 222.0885.

Step 4: 6-Ethyl-1-oxo-indan4-carboxyIic acid (5): The dicarboxylic acid (4) (1.0 g, 4.5 mmol) was thoroughly mixed with anhydrous AlCl₃ (4.2 g, 31.5 mmol) and sodium chloride (0.7 g, 12.1 mmol). The solid was heated with occasional stirring for 2 h to ca. 160° C., resulting in a dark, viscous oil. After cooling, the complex was hydrolysed by stirring for 8 h with ice water (4 ml) and 6N HCl (12 ml). The solid product (5) was collected by filtration, washed thoroughly with water and dried; Yield: 0.66 g (71%). M.p.: 172° C.

¹H NMR ([D₆] DMSO, 500 MHz): δ=1.22 (t, 3H), 2.63 (t, 2H), 2.73 (q, 2H), 3.32 (t, 2H), 7.65 (s, 1H), 8.05 (s, 1H), 13.03 (s).-¹³C NMR ([D₆] DMSO, 125 MHz): δ=15.3 (CH₃), 26.6 (CH₂), 27.4 (CH₂), 36.0 (CH₂—CO), 125.8 (Cₐᵣₒₘ—COOH), 128.8 (CH_{arom ),} 135.8 (CHₐᵣₒₘ), 138.2 (CHₐᵣₒₘ—CO), 143.6 (Cₐᵣₒₘ—C₂H₅), 153.9 (Cₐᵣₒₘ—CH₂), 166.9 (COOH), 205.8 (CO).-IR (KBr): v=2966, 2931, 2871, 2579, 1715, 1675, 1581, 1432, 1303, 1234, 1124, 906, 827 cm⁻1.-MS (70 eV); m/z (%): 204 (100) [M⁺], 189 (31), 186 (24), 176 (46), 161 (29), 143 (16), 133 (27), 115 (26), 91 (16), 77 (14). -HR MS [M⁺]: calcd 204.0786; found 204.0787.

Step 5: 2-[(6-Ethyl-1-oxo-indane-4-carbonyl)-amino]-3-methylentanoic acid methyl ester (6): A chilled and well-stirred solution of 6-ethyl-1-oxo-indan-4-carboxylic acid (5) (0.4 g, 1.96 mmol), hydrochloride of L-isoleucine methyl ester (0.407 g, 2.24 mmol), 2,4,6-collidine (0.63 ml, 4.8 mmol) in dry DMF (25.0 ml) was gradually treated with O-(7-Aza-1-benzotriazolyl)-N,N,N′,N′tetramethyluroniumhexafluoro-phosphate (HATU) (0.81 g, 2.16 mmol). Stirring was continued for 2 h at 0° and for another 10 h at Room temperature. Then, a solution of sat aq. NaHCO₃ (10 ml) was added, and, after 10 min, the mixture was extracted with diethyl ether (3×5 ml). The organic layer was washed with sat. solution of NaCl (5 ml) and dried (MgSO₄). After removal of the solvent in vacuo, the crude isoleucine conjugate was purified by chromatography on silica gel using ethyl acetate:hexane (2:1, v/v) for elution. Yield: 0.56 g, (86%). M.p.: 79° C.

¹H NMR (CDCl₃, 500 MHz): δ=0.92 (t, 3H), 0.94 (d, 3H), 1.21 (t, 3H), 1.47 (m), 1.61 (m), 1.98 (m), 2.69 (q, 2H), 3.30 (m, 1H), 3.73 (s, 3H), 4.78 (dd, 1H), 6.50 (d, NH), 7.20 (s), 7.65 (s).-¹³C NMR (CDCl₃, 125 MHz): δ=11.9 (CH₃), 15.6 (CH₃), 15.8 (CH₃), 25.7 (CH₂), 26.1 (CH₂), 28.6 (CH₂), 36.7 (CH), 38.5 (CH₂), 52.6 (OCH₃), 57.0 (CH), 125.6 (CHₐᵣₒₘ); 133.1 (CHₐᵣₒₘ), 133.3 (Cₐᵣₒₘ), 138.8 (Cₐᵣₒₘ), 144.6 (Cₐᵣₒₘ), 151.5 (Cₐᵣₒₘ—Et), 167.0 (CONH), 172.8 (COOMe), 206.7 (CO).-IR (KBr): v=3335, 3312, 2965, 2923, 2872, 1756, 1738, 1701, 1659, 1585, 1525, 1298, 1196, 1150, 983, 831 cm⁻1-MS (70 eV); m/z (%): 331 (21) [M⁺], 299 (2), 272 (15), 243 (3), 203 (16), 187 (100), 186 (52), 159 (13), 131 (5), 115 (8), 91 (8), 71 (7), 57 (11).-HR MS [M⁺]: calcd 331.1784; found 331.1785.

Crystal Structure Determination: The intensity data for the compound were collected on a Nonius KappaCCD diffractometer, using graphite-monochromated Mo—K_{α} radiation. Data were corrected for Lorentz and polarlsation effects, but not for absorption. The structure was solved by direct methods (SHELXS) and refined by full-matrix least squares techniques against Fo² (SHELXL-97). The hydrogen atoms of the structure were included at calculated positions with fixed thermal parameters. All non-hydrogen atoms were refined anisotropically. XP (SIEMENS Analytical X-ray Instruments, Inc.) was used for structure representations.

Crystal Data for conjugate (6): C₁₉H₂₅N O₄, Mr=331.40 gmol⁻1, colourless prism, size 0.18×0.12×0.10 mm³, monoclinic, space group P2₁, a=8.7528(4), b=22.2904(9), c=10.0250(3) Å, β3=115.620 (2)°, V=1763.6(1) A³, T=−90° C., Z=4, _{ρcalcd}.=1.248 gcm³, μ (Mo—K₆₀)=0.87 cm⁻1, F(000)=712, 6577 reflections in h(−11/11), k(−26/28), I(−11/11), measured in the range 3.77*≦Θ≦27.50°, completeness Θ max=95.7%, 6577 independent reflections, 5495 reflections with Fo>4σ(Fₒ), 441 parameters, 1 restraints, R1_{obs}=0.057, wR²_{obs}=0.102, R1ₐₗₗ=0.075, wR². ₁₁=0.108, GOOF=1.049, Flack-parameter 0.2(9), largest difference peak and hole: 0.171/−0.191 e Å⁻3.

### Example 2

### Preparation of 6-Methoxy-1-oxo-indanoyl-L-isoleucine Alkyl Esters 12a-d

Step 1: 2-(2-Carboxy-ethyl)-5-methoxy-benzoic acid (8): A mixture of 6-methoxy-1,2-dihydronaphthalene (0.97 g, 6.1 mmol), sodium periodate (5.5 g, 25.7 mmol), ruthenium trichloride hydrate (30 mg, 2.2 mol %), acetonitrile (12 mL), CCl₄ (12 mL), and water (18 mL) was stirred magnetically for 2 h. The temperature, which was initially room temperature, rose during the reaction. The reaction mix was extracted several times with dichloromethane and filtered. The solvent was removed under low pressure to give an oil, which was dissolved in 10 mL dioxane/water (1/1, v/v). To this mixture was added aq AgNO₃ (2 g in 10 mL) and aq KOH (3 g in 10 mL). Stirring at RT for 1 d, filtering through celite 545, acidification with conc. HCl, and extraction with ethyl acetate gave the product as a tan solid. Yield: 0.75 (55%). Mp(decomp): 174° C. ¹H NMR ([D₆] DMSO): □=2.48 (t, J=7.7 Hz, 2H), 3.07 (t, J=7.7, 2H), 3.77 (s, 3H), 7.05 (dd, J=8.5 Hz, J=3.0 Hz, ₁H), 7.25 (d, J=8.5 Hz, 1H), 7.31 (d, J=2.9 Hz, 1H). ¹³C NMR ([D₆]DMSO): δ=28.34, 35.60, 55.22, 115.05, 117.63, 131.33, 132.02, 133.72, 157.32, 168.42, 173.83. IR (KBr): v=3700-2400br., 2940, 2625, 1690, 1614, 1571, 1500, 1419, 1310, 1234, 1076, 1044, 892, 821, 765, 685, 538. MS (70 eV): m/z (%)=224(24), 206(11), 178(100), 165(57), 161(14), 149(18), 135(12), 121(7), 109(9). HR-MS [M⁺]: calcd. 224.068474; found 224.068367. Step 2: Intramolecular Friedel-Crafts Acylation and Esterification: General Procedure for esters 10b-d: A) The dicarboxylic acid 8, AlCl₃ (28.0 g) and NaCl (4.0 g) were thoroughly mixed and placed in 250 mL round bottomed flask covered by an Ar atmosphere. The mixture was heated to 140° C. for 3 h with occasional manual stirring and releasing of gas pressure. After coolirig, the flask was chilled and ice (ca. 100 g), followed by conc. HCl (25 mL) were slowly added. Stirring was continued for 6 h and the product was extracted with ethyl acetate. After drying (MgSO₄) the solvent was removed i.v. to give 9 as a brown solid. B) The crude product was suspended in DMF (40 mL), and an appropriate base (K₂CO₃, CSCO₃) along with an alkyl bromide with Kl, or a sulfonate were added. The mixture was sonicated or stirred until reaction was complete (TLC). The reaction mix was poured into ether and washed several times with water and sat aq NaCl. After drying over MgSO₄, the solvent was removed i.v. and the product purified by flash chromatography.

Step 3: Methyl 6-methoxy-1-oxo-indan4-carboxylate (10a): Prepared from 8 (1.00 g, 4.5 mmol) as described above. Alkylation was achieved in the presence of K₂CO₃′ (1.68 g) and dimethyl sulfate (4 mL, 42 mmol). After stirring for 1 d, the product was worked up and purified by flash chromatography using petrol ether/ethyl acetate (2:1, v:v) for elution. Crystallization from petrol ether:ethyl acetate provided slightly yellow needles of 10a. Yield: 0.204 g (21%). Mp: 130.0-131.0° C. ¹H NMR ([D₆]DMSO): δ=2.65 (m, 2H), 3.26 (m, 2H), 3.85 (s, 3H); 3.87 (s, 3H), 7.33 (d, J=2.3 Hz, 1H), 7.69 (d, J=2.8 Hz, 1H). ¹³C NMR ([D₆]DMSO): δ=26.02, 36.14, 52.23, 55.91, 110.21, 123.16, 128.61, 139.43, 148.08, 158.83, 165.31, 205.47. IR (KBr): v=3071, 3018, 2973, 2952, 2844, 1717, 1435, 1326, 1233, 1123, 1066, 1035, 779. MS (70 eV): m/z (%)=220(100), 205(78), 188(31), 177(14), 161(39), 133(15). HR-MS[M⁺]: calcd. 220.073559; found 220.073559.

Step 4: Propyl 6-Dropoxy-1-oxo-indan-4-carboxylate (10b): Prepared from 8 (1.04 g, 4.5 mmol) as described above. Alkylation was achieved in the presence of Cs₂CO₃ (1.68 g) and 1-bromopropane (5 mL, 55 mmol). After sonication until completion (TLC), the product was worked up and purified by flash chromatography using petrol ether:ethyl acetate (2:1, v:v) for elution. Yield: 0.42 g (33%). Orange crystals. Mp: 44.647;1° C. ¹H NMR (CDCl₃): δ=1.04 (m, 6H), 1.82(sept, J=7.2 Hz, 4H), 2.70 (m, 2 H), 3.38 (m, 2H), 3.97 (t, J=6.4 Hz, 2H), 4.29 (t, J=6.6 Hz, 2H), 7.36 (d, J=2.8 Hz, 1H), 7.84 (d, J=2.8 Hz, 1H). ¹³C NMR (CDCl₃): δ=10.54, 10.75, 22.21, 22.51, 26.60, 36.73, 66.97, 70.37, 110.83, 125.37, 129.31, 139.66, 148.64, 158.92, 165.79, 206.60. IR (KBr): v=3077, 2962, 2929, 2874, 1721, 1700, 1607, 1470, 1312, 1230, 1208, 1120, 1060, 902, 776. MS (70 eV): nmlz (%)=276(84), 248(32), 233(40), 217(20), 192(99), 191(100), 174(16), 164(20), 147(28), 119(16). HR-MS[M⁺]: calcd. 276.136159; found 276.136238.

Step 5: Allyl 6-allyloxy-1-oxo-indan-4-carboxylate (10c ): Prepared from 8 (1.00 g, 4.5 mmol) as described above. Alkylation was achieved in the presence of Cs₂ CO₃ (3.0 g) and allyl bromide (5 mL, 61 mmol). After sonication until completion (TLC), the product was worked up and purified by flash chromatography using petrol ether:ether (2:1, v:v) giving the product as a faint yellow oil. Yield: 0.214 g (18%). ¹H NMR (CDCl₃): δ=2.72 (m, 2H), 3.41 (m, 2H), 4.60 (t, J=1.5 Hz, 1H), 4.62 (t, J=1.5 Hz, 1H), 4.85 (t, J=1.4 Hz, 1H), 4.86 (t, J=1.4 Hz, 1H), 5.31 (qnt, J=1.4 Hz, 1H), 5.33(qnt, J=1.4 Hz, 1H), 5.42 (sxt, J=1.5 Hz, 1H), 5.45 (sxt, J=1.5 Hz, 1H), 6.05 (m, 2H), 7.40 (d, J=2.4 Hz, 1H), 7.91 (d, J=2.4 Hz, 1H). ¹³C NMR (CDCl₃): δ=26.59, 37.00, 65.93, 69.44, 111.49, 118.38, 118.90, 125.42, 129.02, 132.01, 0.132.43, 139.72, 149.07, 158.32, 165.21, 206.40. IR (NaCl): v=3086, 3021, 2987, 2935, 2875, 1717, 1610, 1580, 1476, 1420, 1313, 1227, 1128, 1059, 1024, 930. MS (70 eV): m/z (%)=272(35), 231(100), 215(11), 191(7), 146(6), 89(7). HR-MS[M⁺]: calcd. 272.104859; found 272.104568.

Step 6: Pentyl 6-pentoxy-1-oxo-indancarboxylate (10d): Prepared from 8 (1.00 g, 4.5 mmol) as described above. Alkylation was achieved in the presence of Cs₂CO₃ (5.3 g) and n-pentyl bromide (4.5 mL, 36 mmol). After sonicafion to completion (TLC), the product was worked up and purified by flash chromatography using petrol ether:ether (2:1, v:v) for elution. Yield: 0.48 g (32%). Orange solid. Mp: 45.3-48.0° C. ¹H NMR (CDCl₃): δ=0.93(2t, J=6.7 Hz, 6H), 1.41 (m, 8H), 1.80 (m, 4H), 2.71 (m, 2H), 3.38 (m, 2H), 4.01 (t, J=6.6 Hz, 2H), 4.33 (t, J=. 6.7 Hz, 2H), 7.36 (d, J=2.4 Hz, 1H), 7.84 (d, J=2.4 Hz, 1H). ¹³ C NMR (CDCl₃): δ=14.08, 14.10, 22.45, 22.53, 26.61, 28.23, 28.35, 28.54, 28.86, 36.76, 65.53, 68.92, 110.75, 125.39, 129.34, 139.68, 148.66, 158.95, 165.81, 206.64. IR (KBr): v=3074, 2957, 2935, 2868, 1715, 1609, 1482, 1326, 1226, 1130, 1066, 1027, 899, 722, 583. MS (70 eV): m/z (%)=332(63), 262(24), 261(21), 245(11), 192(100), 174(10), 164(5)147(15), 119(7). HR-MS[M⁺]: calcd. 332.198760; found 332.198021.

Step 7: 6-Methoxy-1-oxo-indancarboxylic acid (11a): A chilled solution of 10a (0.165 g, 0.75 mmol) in methanol (25 mL) was gradually treated with aqueous KOH (20% soln. in water, 25 mL). Stirring was continued at rt for 50 min. The solvent was then reduced i.v. to ca. 30% with gentle warming (35° C.). Unreacted ester was extracted with ethyl acetate. Following acidification with conc. HCl, the acid was extracted with ethyl acetate. The organic extract was washed with water and sat. NaCl solution. After removal of solvent, the product was adsorbed onto celite 545 and purified by flash chromatography, using petrol ether:ether (2:1, v:v, with 5% acetic acid) for elution. The pure acid was obtained an off-white solid. Yield: 0.101 g (65%). Mp (decomp): 220° C. ¹H NMR ([D₆DMSO): δ=2.68 (m, 2H), 3.31 (m, 2H), 3.88 (s, 3H), 7.33 (d, J=2.4 Hz, 1H), 7.73 (d, J=2.8 Hz, 1H). C NMR ([De]DMSO): δ=26.16, 36.20, 55.80, 109.60, 123.40, 130.16, 139.27, 148.30, 158.78, 166.58, 205.70. IR (KBr): v=3600-2750br., 3123, 3072, 2973, 2848, 1711, 1685, 1481, 1436, 1333, 1038, 694. MS (70 eV): m/z (%)=206(100), 188(7), 178(15), 161(40), 149(12), 135(19), 121(10). HR-MS[M⁺]: calcd. 206.057909; found 206.057808.

Step 8: 6-Pronoxy-1-oxo-indan4-carboxylic acid (11b): A chilled solution of 10b (0.152 g, 0.61 mmol) in methanol (25 mL) was gradually treated with aqueous KOH (20% soln. in water, 25 mL). Stirring was continued at rt for 3 h. The solvent was then reduced i.v. to ca. 30% with gentle warming (35° C.). Unreacted ester was extracted with ether. Then, the aqueous phase was acidified with conc. HCl and the free acid was extracted with ether. After drying (MgSO₄), the product was adsorbed onto celite 545 and purified by flash chromatography using petrol ether:ether (2:1, v:v with 5% acetic acid) for elution. The pure acid was obtained as an off-white solid. Yield: 0.090 g (70 %). Mp: 211.5-213.3. ¹H NMR ([D₆]DMSO): δ 0.98 (t, J=7.5 Hz, 3H), 1.73 (sxt, J=7.0 Hz, 2H), 2.63 (m, 2H), 3.26 (m, 2H), 4.00 (t, J=6.4 Hz, 2H), 7.26 (d, J=2.4 Hz, 1H), 7.67 (d, J=2.4 Hz, 1H). ¹³C NMR (ID₆]DMSO): 3=10.27, 21.88, 26.15, 36.19, 69.71, 110.25, 123.86, 129.79, 139.29, 148.21, 158.15, 166.50, 205.64. IR (KBr): v=3700-2400br., 3171, 3088, 2971; 2943, 2882, 1724, 1691, 1580, 1307, 1201, 1135, 823, 695. MS (70 eV): m/z (%)=234(64), 192(100), 174(21), 164(20), 147(25), 121(11). HR-MS[M+]: calcd. 234.089209; found 234.089191.

Step 9: 6-Allyloxy-1-oxo-indan-4-carboxylic acid (11c): A chilled solution of 10c (0.084 g, 0.34 mmol) in methanol (25 mL) was gradually treated with aqueous KOH (20% soln. in water, 25 mL, Stirring was continued at rt for 3 h. The solvent was then reduced i.v. to ca. 30% with gentle warming (35° C.). Unreacted ester was extracted with ether. Then, the aqueous phase was acidified with conc. HCl and the free acid extracted with ether. After drying (MgSO₄), the product was adsorbed onto celite 545 and purified by flash chromatography using petrol etherether (2:1, v:v with 5% acetic acid) for elution. The free acid was obtained as an off-white solid. Yield: 0.050 g (70%). Mp: 193.2-194.8. ¹H NMR ([D₆]DMSO): δ=2.65 (m, 2H), 3.28 (m, 2H), 4.69 (m, 2 H), 5.30 (m, 1H), 5.42 (m, 1H), 6.05 (m, 1H), 7.31 (d, J=2.4 Hz, 1H), 7.73 (d, J=2.4 Hz, 1 H). ¹³C NMR ([D₆]DMSO): δ=26.18, 36.20, 68.76, 110.80, 117.65, 124.06, 129.84, 133.18, 139.29, 148.47, 157.64, 166.49, 205.64. IR (KBr): v=3700-2300br., 3148, 3082, 2924, 2585, 1721, 1683, 1610, 1579, 1487, 1426, 1306, 1200, 1137, 1025, 940, 705. MS (70 eV): m/z (%)=232(100), 217(14), 204(21), 187(11), 177(8), 159(25), 145(11). HR-MS[M⁺]: calcd. 232.072559; found 232.073643.

Step 10: 6-Pentoxy-1-oxo-indan-4-carboxylic acid (1.1d): A chilled solution of 10d (0.103 g, 0.37 mmol) in ethanol (25 mL) was gradually treated with aqueous KOH (20% soln. in water, 5 mL). Stirring was continued at rt for 4 h. The solvent was then reduced to ca. 30% with gentle stream of Ar. Unreacted ester was extracted with ether. Then, the aqueous phase was acidified with conc. HCl and the free acid extracted with ether. After drying (MgSO₄), the product was adsorbed onto celite 545 and purified by flash chromatography using petrol ether:ether (3:1, v:v with 5% acetic acid) for elution. The free acid was obtained as an off-white solid. Yield: 0.054 g (67%). Mp: 165.4-166.2° C. ¹H NMR ([D₆]DMSO): δ=0.89 (t, J=7.1 Hz, 3H), 1.36 (m, 4 H), 1.72 (qnt, J=7.0 Hz, 2H), 2.64 (m, 2H), 3.28 (m, 2H), 4.05 (t, J=6.4 Hz, 2H), 7.28 (s, 1H), 7.69 (s, 1H). ¹³C NMR ([D₆]DMSO): δ=13.88, 21.83, 26.14, 27.58, 28.15, 36.19, 68.25, 110.27, 123.87, 129.88, 139.30, 148.20, 158.16, 166.52, 205.66. IR (KBr): v=3700-2400br., 3169, 3082, 2962, 2935, 2874, 2590, 1721, 1689, 1612, 1579, 1481, 1432, 1306, 1230, 1197, 1137, 825, 694. MS (70 eV): m/z (%)=262(43), 192(100), 174(16), 164(15), 147(15), 121(7). HR-MS[M⁺]: calcd. 262.120509; found 262.120438.

For the conversion of 11a-d into the isoleucine methyl conjugates 12a-d below, the method of step 5 of example 1 was applied.

6-Methoxy-1-oxo-indanoyl-L-isoleucine methyl ester (12a): Prepared from 11a (0.077 g, 0.37 mmol) in DMF (5 mL) as described. The product was purified by chromatography on silica gel using petrol ether:ethyl acetate (2:1, v:v) for elution. Yield: 0.097 g (78%). ¹H NMR (CDCl₃): δ=0.95 (m, 6H), 1.25 (m, 1H), 1.51 (m, 1H), 2.01 (m, 1H), 2.70 (t, J=5.6 Hz, 2H), 3.26 (m, 2H), 3.77 (s, 3H), 3.85 (s, 3H), 4.79 (dd, J=8.4 Hz, J=4.7 Hz, 1H), 6.61 (d, J=8.2 Hz, 1H), i. 28 (d, J=2.1 Hz, 1H), 7.44 (d, J=2.2 Hz, 1H). ¹³C NMR (CDCl₃): δ=11.61, 15.57, 25.34, 25.37, 36.63, 38.14, 52.18, 55.89, 56.78, 108.00, 121.90, 133.69, 139.58, 146.08, 159.41, 160.61, 166.25, 172.37, 206.24. IR (NaCl): v=3338, 3061, 2964, 2936, 2877, 2842, 1744, 1716, 1651, 1480, 1338, 1207, 1149, 772. MS (70 eV): m/z (%)=333(40), 274(14), 216(5), 205(23), 189(100), 188(75), 161(31), 133(13), 116(25). HR-MS[M⁺]: calcd. 333.157623; found 333.157616.

6-Propoxy-1-oxo-indanovl-L-isoleucine methyl ester (12b): Prepared from 11b (0.155 g, 0.66 mmol) in DMF (10 mL) as described. The product was purified by chromatography on silica gel using petrol etherethyl acetate (2:1, v:v) for elution. Yield: 0.239 g (84%). Colourless solid. Mp: 97.8-98.9° C. ¹H NMR (CDCl₃): δ=0.97 (m, 6H), 1.04 (t, J=7.3 Hz, 3H), 1.26 (m, 1H), 1.52 (m, 1H), 1.82(sxt, J=7.0 Hz, 2H), 2.02 (m, 1H), 2.71 (t, J=5.8 Hz, 2H), 3.31 (m, 2H), 3.78 (s, 3H), 3.97 (t, J=6.6 Hz, 2H), 4.81 (dd, J=8.6 Hz, J=4.6 Hz, 1H), 6.58 (d, J=8.3 Hz, 1H), 7.28 (d, J=2.4 Hz, 1H), 7.45 (d, J=2.4 Hz, 1H). ¹³C NMR (CDCl₃): 3=10.56, 11.78, 15.72, 22.52, 25.50, 25.52, 36.80, 38.32, 52.44, 56.91, 70.41, 108.64, 122.45, 133.76, 139.70, 146.17, 159.06, 166.47, 172.56, 206.51. IR (KBr): v=3227, 3070, 2966, 2937, 2883, 1745, 1706, 1632, 1558, 1258, 1154. MS (70 eV): m/z (%)=361(45), 302(12), 233(25), 217(90), 216(100), 188(39), 147(26), 119(22). HR-MS[M⁺]: calcd. 361.188923; found 361.189049.

6-Allyloxy-1-oxo-indanoyl-L-isoleucine methyl ester (12c): Prepared from 11c (0.025 g, 0.11 mmol) in DMF (2 mL) as described. The product was purified by chromatography on silica gel using petrol ether:ethyl acetate (2:1, v:v) for elution. Yield: 0.027 g (70%). Colourless solid. Mp: 95.6-96.3° C. ¹H NMR (CDCl₃): δ=0.99 (m, 6H), 1.26 (m, 1H), 1.52 (m, 1H), 2.02 (m, 1H), 2.73 (m, 2H), 3.33 (m, 2H), 3.79 (s, 3H), 4.60 (d, J=5.2 Hz, 2H), 4.81 (dd, J=8.4 Hz, J=4.8 Hz, 1H), 5.32 (d, J=10.4 Hz, 1H), 5.43 (d, J=16 Hz, 1H), 6.05 (m, 1H), 6.55 (d, J=7.5 Hz, 1H), 7.31 (d, J=2.5 Hz, 1H), 7.50 (d, J=2.5 Hz, 1H). ¹³C NMR (CDCl₃): δ=10.79, 14.74, 24.51, 24.56, 36.81, 37.35, 51.47, 55.93, 68.52, 108.17, 117.61, 121.56, 131.41, 132.87, 138.76, 145.49, 157.49, 165.38, 171.54, 205.36. IR (KBr): v=3227, 3075, 2971, 2927, 1745, 1706, 1637, 1607, 1558, 1341, 1292, 1255, 1200, 1150, 1026. MS (70 eV): m/z (%)=359(54), 300(13), 258(7), 231(15), 215(100), 214(95), 186(24), 146(14). HR-MS[M⁺]: calcd. 359.173273; found 359.173295.

6-Pentoxy-1-oxo-indanoyl-L-isoleucine methyl ester (12d : Prepared from 11d (0.034 g, 0.13 mmol) in DMF (2 mL) as described. The product was purified by chromatography on silica gel using petrol ether:ethyl acetate (2:1, v:v) for elution. Yield: 0.040 g (79%). Colourless solid. Mp: 100.6-101.7° C. ¹H NMR (CDCl₃): δ=0.97 (m, 9H), 1.26 (m, 1H), 1.40 (m, 4H), 1.51 (m, 1H), 1.79 (m, 1H), 2.02 (m, 1H), 2.70 (t, J=5.5 Hz, 2H), 3.29 (m, 2H), 3.77 (s, 3H), 4.00 (t, J=6.4 Hz, 2H), 4.80 (dd, J=8.2 Hz, J=4.6 Hz, 2H), 6.59 (d, J=8.2 Hz, 1H), 7.27 (s, 1H), 7.44 (s, 1H). ¹³C NMR (CDCl₃): δ=11.96, 14.29, 15.91, 22.71, 25.69, 25.70, 28.41, 29.05, 36.98, 38.50, 39.73, 52.62, 57.11, 69.10, 108.81, 122.67, 133.94, 139.99, 146.35, 159.24, 166.67, 172.74, 196.70, 206.70. IR ((KBr): v=3242, 3060, 2966, 2937, 2878, 1750, 1701, 1640, 1607, 1550, 1341, 1297, 1258, 1198, 1149, 888. MS (70 eV): m/z (%)=389(54), 330(11), 261(20), 245(78), 244(100), 216(30), 191(12), 175(12), 146(18), 145(19), 119(13). HR-MS[M⁺]: calcd. 389.220223; found 389.219559.

### Example 3

### Collection of Volatiles

Plant material: Induction experiments were performed with plantlets of the Lima bean Phaseolus lunatus (Ferry Morse cv. Jackson Wonder Bush). Individual plants were grown from seed in a plastic pot (Ø=5.5 cm) at 23° C. and 80% humidity using daylight fluorescent tubes at ca. 270 μE m² st⁻1 with a photophase of 14 h. Experiments were conducted with 12-16 day-old seedlings showing two fully developed leaves.

Induction experiments: Plantlets of P. lunatus with two fully developed primary leaves were cut with razor blades and immediately transferred into Eppendorf vials containing a solution of the test-substance in tap water (100 μl). Solutions of the unsubstituted prior art compound indanoyl-L-isoleucine and of 6 -ethyl indanoyl-L-isoleucine methyl ester according to the invention were applied at 10, 100 and 1000 μM. Coronatne was used as a 100 μM aqueous solution. After uptake of the solution, the plantlets were placed into vials with tap water (5 ml) and were enclosed in small desiccators (750 ml) and maintained at 25° C. for 24 h. Control experiments were conducted under identical conditions by placing freshly cut plantlets into tap water. All experiments were carried out in triplicate.

Collection and analysis of headspace volatiles: The volatiles emitted from the pre-treated plants were collected continuously on small charcoal traps (1 mg charcoal, CLSA-Filter, Le Ruisseau de Montbrun, F-09350 Daumazan sur Arize, France) over a period of 24 h using air circulation as described in J. Donath and W. Boland, Phytochemistry 1995, 39, 785-790.

After desorption of the volatiles from the carbon trap with 40 μl of a solution of 1-bromodecane (internal standard, 50 μM) in dichloromethane. The extracts were directly analysed by GC/MS. GC-conditions: Fused-silica capillary (30 m×0.25 mm) coated with DB 5 (0.25 μm). Helium at 40 cm min⁻1 served as carrier gas. Separation of the compounds was achieved under programmed conditions (50° C. for 2 min, then at 10° C. min⁻1 to 200° C., finally at 35° C. min⁻1 to 280° C.). MS: Finnigan GCQ; GC-interface at 265° C.; scan range 35-450 Da. Individual compounds (peak area) were quantified with respect to the peak area of the internal standard.

### Example 4

Tendril coiling: To test the ability of the conjugates to induce tendril coiling, shoots of Bryonia dioica with the youngest, most well-developed tendrils were cut and immediately placed into vials with the test solution (2 ml) with the indanoyl conjugates (5-1000 μM) or coronatine (5-100 μM) and the extent of coiling was followed over a period of 20 h. The efficacy of the compounds as to their ability to induce tendril coiling was evaluated by the experimentator accordihg to the following scale:

| | |
|---|---|
| − | no coiling |
| + | minor coiling |
| ++ | Considerable coiling |
| +++ | intense coiling |

From the following table, the advantageous properties of the 6-substituted indanoyl isoleucine derivative as compared to the unsubstituted prior art compound are apparent. Also, the compound according to the invention shows improved properties as compared to the naturally occurring compound coronatine:

| | tendril | tendril | tendril |
|---|---|---|---|
| | coiling | coiling | coiling |
| | at 500 μm | 100 μm | 10 μm |
| Elicitor | concentration | concentration | concentration |
| 1-oxo-indanoyl | ++ | + | − |
| isoleucine methyl ester | | | |
| (prior art | | | |
| compound) | | | |
| 1-oxo-6-ethyl indanoyl | +++ | +++ | + |
| isoleucine methyl ester | | | |
| coronatine | +++ | +++ | ++ |

1. A compound of the chemical formula (I) 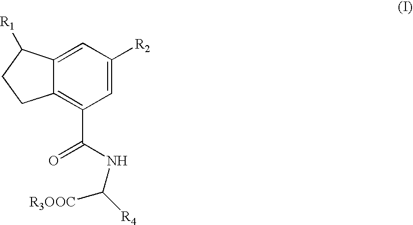
wherein
R₁= 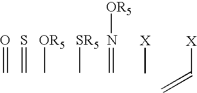
R₂=linear or branched C₁-C₈-alkyl, -alkenyl, or -alkinyl;
saturated or unsaturated, linear or branched C₁-C₈-ether or -polyether or 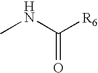
R₃=H or a residues that forms an ester which can be easily saponified by the plant, R₄=a side chain of an L-amino acid;
R₅=H, acyl or linear or branched C₁-C₈-alkyl, -alkenyl, or -alkinyl;
R₆=H or linear or branched C₁-C₈-alkyl, -alkenyl, or -alkinyl; or
a saturated or unsaturated, linear or branched C₁-C₈-ether or -polyether.

2. The compound according to claim 1, wherein R₃ is a linear or branched C₁C₄-alkyl, -alkenyl, or -alkinyl; benzyl, phenyl or allyl.

3. The compound according to claims 1 or 2, wherein R₄ is defined as the side chain of an aliphatic or isocyclic amino acid.

4. The compound according to claims 1 to 3, wherein R₄ is defined as isoleucin, leucin, allo-isoleucin, norvalin, norleucin or coronamic acid.

5. The compound according to claims 1 to 4, wherein
R₁ is ═O,
R₂ is —CH₂—CH₃₁
R₃ is CH₃ and
R₄ is the side chain of isoleucine.

6. A process for producing a compound of formula (I) according to claim 1 comprising the reaction of an 6-substituted 1-oxo-indan4-carboxylic acid with the following structural formula (II) 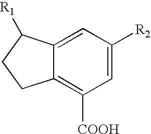
wherein
R₁= 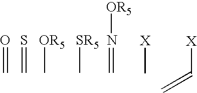
and
X=a halogen atom;
R₂=linear or branched C₁-C₈-alkyl, -alkenyl, -alkinyl;
saturated or unsaturated, linear or branched C₁-CB-ether or -polyether or 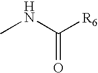
R₅=H, acyl, linear or branched C₁-C₈-6-alkyl, -alkenyl, or -alkinyl;
R₆=H or linear or branched C₁-C₈-alkyl, -alkenyl, -alkinyl; or
a saturated or unsaturated, linear or branched C₁-C₈-ether or -polyether
with an L-amino acid or amino acid ester.

7. The process according to claim 6, wherein the reaction is carried out in a mixture of DMF and collidine in the presence of (O-(7-Aza-1-benzotriazolyl)-N,N,N′,N′-tetramethyluroniumhexafluoro-phosphate (HATU).

8. The process according to claims 6 or 7, wherein an aliphatic or isocyclic L-amino acid is used.

9. The process according to claims 6 to 8, wherein isoleucin, leucin, allo-isoleucin, norvalin, norleucin or coronamic acid or its biochemical precursor or an ester of any of the mentioned amino acids is used as one of the educts.

10. The process according to any of claims 6 to 9, further comprising the steps of
a) oxidauvely cleaving the non-aromatic double bond of 6-methoxy-1,2 dihydronaphtalene (7) 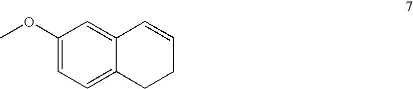
to yield a dicarboxylic acid (8) 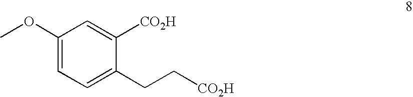
b) conversion of (8) via intramolecular Friedel-Crafts acylation to give a 6substiuted indanone derivative (9) as an intermediate, 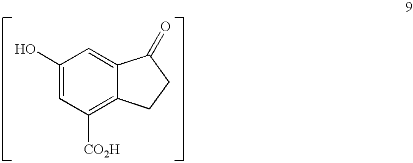
c) and formation of an ether of the 6-hydroxy group of the substituted indanone (9) with RX, wherein X is a suitable leaving group and R is a saturated or unsaturated, linear or branched C1-C8 residue, optionally interrupted by further O atoms, to yield a compound of formula (II), wherein R₁ is ═O and R₂ is a saturated or unsaturated, linear or branched C₁-C₈-ether or -polyether

11. The process according to any of claims 6 to 9, further comprising the Steps of
a) reacting tetrahydronaphthalin in the presence of AlCl₃ and an acyl halide R′—C(O)X; wherein R is hydrogen or a linear or branched C₁-C₇alkyl, alkenyl, or alkinyl residue, to form a diketone (2) 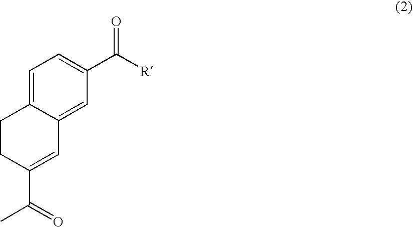
b) oxidative cleavage of (2) at the non-aromatic double bond to yield the triketone intermediate (2′) 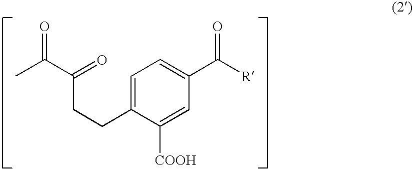
which is rapidly further oxidised to give the dicarboxylic acid (3) 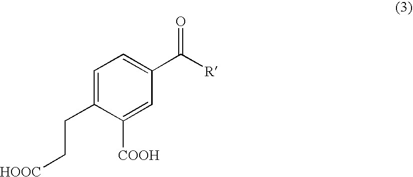
c) reduction of (3) to yield the aromatic dicarboxylic acid (4) 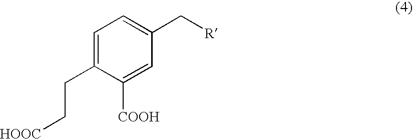
d) and effecting an intramolecular Friedel-Crafts acylation on (4) to yield a compound of formula. (II), wherein R₁ is ═O and R₂ is a linear or branched C₂-C₈-alkyl, alkenyl, or alkinyl residue.

12. The process of claim 10 or 11, further comprising the step of substituting the keto functional group in the indanone part of compound (II) to yield an R₁ chosen from 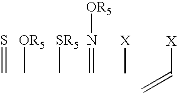
with X being a halogen atom, poor to the reaction of the compound of formula (II) with the amino acid.

13. A composition comprising a compound of any one of claims 1 to 5.

14. A plant protection agent comprising a compound of any of claims 1 to 5.

15. The composition according to claim 13 or the plant protection agent according to claim 14 further comprising an insecticide, a growth regulation agent, a herbicide, a fungicide and/or a fertiliser.

16. The composition according to claims 13 or 15 or the plant protection agent according to claims 14 or 14 which is in the form of a powder, a suspension, dispersion, emulsion, paste or granulate.

17. Use of a compound of any one of claims 1 to 5 or of a composition or plant protection agent of any one of claims 13 to 16 for treating plants to induce a resistance against pathogens.

18. The use according to claim 17, wherein the pathogen is selected from the group consisting of harmful bacteria, fungi, viruses, insects and nematodes is induced.

19. A method for treating a plant to induce a resistance against pathogens, wherein the composition according to claims 13 or 15 or the plant protection agent according to claims 14 or 15 is applied to the plant.

20. The method according to claim 19, wherein the plant protection agent is in the form of a powder, a suspension, dispersion, emulsion, paste or granulate, which is continuously distributed or dispersed onto the plant or its root in accordance with the respective purpose.

21. Use of a compound of any one of claims 1 to 5 or of a composition of claim 13, 15 or 16 for inducing in plants senescence selectively in fruit.

22. A method for selectively inducing senescence in fruit of plants comprising the step of applying a compound of any one of claims 1 to 5 or a composition of claim 13, 15 or 16 to a plant.
